(19) United States
(12) Patent Application Publication
(10) Pub. No.: US 2002/0007497 A1
(43) Pub. Date: Jan. 17, 2002

(54) Food product and process

(76) Inventor: Corran Norman Stuart McLachlan, North Shore (NZ)

Correspondence Address: YOUNG & THOMPSON, 745 SOUTH 23RD STREET 2ND FLOOR, ARLINGTON, VA, 22202

(21) Appl. No.: 09/906,614

(22) Filed: Jul. 18, 2001

Related U.S. Application Data
(63) Continuation of application No. 09/500,801, filed on Feb. 10, 2000.

(30) Foreign Application Priority Data
May 16, 1995 (NZ) 272133

(51) Int. Cl. A01K 67/027

(52) U.S. Cl. 800/15

(57) ABSTRACT

A milk or other dairy product, capable of minimising the onset of disease such as coronary heart disease or enhancing the immune response is derived from animals which are substantially free of the β-casein A¹ allele. Bulk milk can be produced by testing for and culling cows who test positive for the β-casein A¹ allele, or by producing immunoglobulins and other immune response proteins, in cow's milk from animals not possessing the β-casein A¹ allele, or other commercial milk producing animals, to this allele, to counteract the immunosuppressant substances present that are produced from it, in commercial milking cows such as Holsteins, together with its blending with non-treated milk or the recovery of such immunoproteins.

### FIELD OF THE INVENTION

This invention relates to the removal, or the production of immunoglobulins against, immunosuppressant substances present, or, produced from the milk of animals of the genus Bos, and more particularly the domestic dairy species of the group Bos taurus and their crosses with the group Bos Indicus, which are used for milk production, and which contain specific casein alleles.

### BACKGROUND OF THE INVENTION

### Description of the Background Art

It has long been understood that the early lactation mammary secretions of certain species, known as colostrum, contains substances that prevent disease, whilst the immune system of the young of the species is developing. This is particularly true of the ruminants, such as the Bos family. However the ingestion of colostrum is not essential in the human. These substances were identified as proteins (globulins) with immuno-properties which became known as immunoglobulins, (B L Larsen Immunoglobulins of Mammary Secretions in Advanced Dairy Chemistry Volume 1 Proteins. Ed. P F Fox Elsevier, 1992).

Immunoglobulins are present in the serum and mammary secretions of all mammalian species as part of the immune defence system of the animal. The immunoglobulins are also known as antibodies and are produced by the body's immune system in response to the presence of substances called antigens, including a wide range of molecules, bacteria, viruses, cells and particles that do not express specific markers of ‘self’ called histocompatibility antigens. Molecular antigens are largely peptides, proteins and carbohydrates. The classic immune response involves the production of antibodies capable of neutralising these antigens.

The term antigen is now widely used to indicate any molecule that can be specifically recognised by the adaptive elements of the immune system, that is by both B cells, which produce immunoglobulins and T cells which release substances such as cytokines, (Immunology, 3rd Edition, Ed. I Roitt, J Brostoff, D Male, Mosby, London, 1993).

There are five classes, or isotypes, of immunoglobulins all of which have a similar basic structure, but have differences in their organisational structure as well as the amino acid sequences present and carbohydrate groups present. In addition to the immunoglobulins there are present related immune system proteins. These are known as complement and they are a complex group of proteins which assist the function of antibodies. Their properties are described in the above texts. There are at least 11 proteins in the complement group some of which are expected to be present in milk at the milligram per 100 milliliter level.

There are numerous patents that have been filed which seek to:

1. isolate the immunoglobulins present in mammary secretions, particularly colostrum but also including milk and products derived from milk such as whey. Generally the species involved is the domestic cow, Bos taurus, but it may include sheep or goats.

2. produce an “immune milk” or “health food” incorporating the immunoglobulin proteins, either as a result of stimulating the milk producer's immune system by the addition or injection of substances into the animal's body, either once or systematically, which result in an immune response, or by concentrating the small amounts of immunoglobulins that are naturally present in milk-derived products. In the former case the immunoglobulins may be specific responses to the injection of pathogenic bacteria into the milk-producing animals.

Examples of these patents include:

Japanese patent (1988) JP 63-133941, Hori T, Nishimoto K, Kimura M, Yommazaki N, describes a process in which immunoglobulins are collected by ultrafiltration from whey, the by-product of cheese or casein manufacture. The immunoglobulin content of powder derived from this process was found to contain about ten times the immunoglobulin content of dried human milk.

UK Patent Application (1987) GB 2 179 947 Monsan P F E, Thibault P A, Brossad C, Bruvier C S J describes a process for the extraction of proteins, preferably lactoferrin or immunoglobulins from whey comprising concentration of the whey using ultrafiltration with a polysulphone membrane (with MW cut-off 25,000-50,000) followed by diafiltration. The retentate is then subject to adsorption of the retained proteins by ion exchange treatment using a weak cationic carboxymethyl resin at pH 5-8.5 and preferably at 7-8; and elution at the same pH.

European Patent Application (1984) EP 0 102 831 A¹, Linggood M A, Porter P, Powell J R describes the immunisation of host animals with a range of E. Coli implicated in human gastroenteric disease and the production of immunoglobulins, and a synthetic milk containing the immunoglobulins that are specific responses to the inoculation of the host.

UK Patent Application (1987) 8729031, to R C Bottomley claims the production of a whey protein concentrate rich in immunoglobulins by the use of ultra-filtration through a membrane having a cut-off of 500,000 daltons which retains the immunoglobulins, or by subjecting whey to the action of an anion exchange resin which does not remove immunoglobulins so causing an increase in their concentration in the effluent.

European patent application (1989) EP 0 336 694 Beck L R, describes a process for extracting an anti-inflammatory factor from cow or ewe milk, taken from animals that have been previously immunised by the administration of bacterial antigens. The anti-inflammatory factor is then extracted from whey that has been subjected to ion-exchange chromatography and molecular sieve chromatography.

U.S. Pat. No. (1992) 5,106,618 Beck L R, Kotler D P describes the production of a ‘hyperimmune’ milk obtained by inoculating a milk-producing animal with a non-protozoan bacterial antigens, collecting the milk from the animal and the pasteurising and concentrating prior to use.

U.S. Pat. Nos. (1989) 4,879,110 and (1993) 5,194,255 Beck L R, Stolle R J, describe a method for inducing the production of a milk anti-hypertensive factor in an animal such as a cow by injecting bacterial antigens into the animal. The anti-hypertension factor is isolated by (1) removing from the milk molecules having a molecular weight greater than 10,000 daltons; (2) fractionating by ion-exchange chromatography the effluent to obtain a negatively charged fraction; (3) fractionating the negatively charged material eluted from the ion-exchange column using molecular sieve chromatography and isolating the hypertensive fraction from the latter step by isoelectric precipitation.

U.S. Pat. No. (1980) 4,216,236 Mueller H R, Legier C N, Secretin M C, Blonay C N claims the incorporation of soluble proteins obtained from whey using an ultrafiltration step with membranes having a molecular weight cut-off between 1000 and 500,000 incorporating immunoglobulins or to which immunoglobulin powder or concentrate has been added.

U.S. patent (1984) 4,490,290 Ganni M M, May K, Porter P, describes the recovery of one or more milk immunoglobulins by passing the milk through a re-usable immunoadsorbent column comprising an insoluble carrier material to which is bound a low-affinity monoclonal antibody specific to the antibody(ies) but not specific to any other common constituent of milk. The bound immunoglobulin(s) are released by eluting the immunosorbent with 4M MgCl₂.

Problem

Notwithstanding all these patents and the claimed benefits of their products there is a considerable body of evidence that links milk particularly of the Bos taurus, the domestic cow with allergy problems with young children, asthma, chronic immune disorders such as diabetes mellitis, and atherosclerosis. Recent studies have also linked increased consumption of casein with the formation of hepatic tumours in rats, due it appears to a depressed NK cell cytotoxic activity, Bell R C, et al Nutr Cancr 22:151-162, (1994).

To date it has not been possible to identify any particular fraction or molecule that is responsible for disorders such as atherosclerosis, although the consumption of animal fats and their associated saturated fatty acids have been claimed to either cause, or contribute to, coronary heart disease, hypertension and obesity as is set out in most medical texts on these subjects and the Surgeon-General's Report on Nutrition and Health, DHHS Publication No 88-50210 (1988).

### Object

It is an object of this invention to provide an improved food product and/or process or one which will at least provide the public with a useful choice.

### Definitions

“β-casein A¹ Allele” is a term used herein in reference to one of the variant forms of the β-casein gene. Expression of the A¹ allele results in the production of “β-casein A¹”.

Where reference is made to the presence of the β-casein A¹ allele in an individual or population it encompasses both homozygous and heterozygous genotypes with respect to that allele. Similarly, where reference is made to the presence of β-casein A¹ it encompasses phenotypes resulting from either a homozygous or heterozygous state with respect to the β-casein A¹ allele.

The term “Immune milk” is used herein reference to milk obtained from an animal that has been immunised to selectively induce for formation of immunoglobulins and other immune proteins, directed against specific bacterial and/or viral pathogens or other foreign antigens that are known to cause diseases, in its milk, such milk being used to prevent disease, within the milk drinker, by fortifying the body's natural resistance against specific disease-causing antigens.

This invention is applicable to all products derived from cattle (live or dead) which products are substantially free of β-casein A¹, or contains immune response proteins (including immunoglobulins) to β-casein A¹. This includes meat (including offal) blood and blood products (such as black pudding), casein, gelatin, milk and other dairy products, as well as manufactured products containing some or all of the foregoing examples (including whiteners for beverages that include some milk solids).

The term “processed dairy product(s)” is used herein to refer to dairy products derived from a source of bulk milk (ie from milk from more than one animal) and includes, but is not limited to:

(a) bulk milk used to make cheese whether or not the milk has been pasteurised or sterilised prior to cheese making,

(b) milk powder(s),

(c) milk fats,

(d) milk solids,

(e) casein(s), caseinate(s), and casein hydrolysates,

(f) pasteurised, sterilised, preserved milks including microfiltered milks, UHT milks,

(g) low fat milks,

(h) modified or enhanced milks,

(i) ice-cream or other frozen dairy based confections,

(j) fermented milk products such as yoghurt or quark,

(k) cheeses including full fat, partial de-fatted and fat-free processed cheeses,

(l) milk whey,

(m) food products enriched through the addition of milk products such as soups,

(n) milk from which allergenic molecules have been removed,

(o) confections such as chocolate,

(p) carbonated milk products, including those with added phosphate and/or citrate,

(q) infant formulations which may contain full, partially de-fatted or nonfat milk together with a number of additional supplements,

(r) liquid or powdered drink mixtures,

(s) butter, buttermilk, buttermilk powder.

### STATEMENT OF INVENTION

In one aspect the invention provides a food product derived from animals which product is substantially free of the β-casein A¹ allele, or β-casein A¹ expressed therefrom.

Preferably the food product is meat or meat related, and more preferably is derived from an animal which is substantially free of the β-casein A¹ allele.

In a particularly preferred form of the invention the food product is milk or other dairy product which is substantially free of β-casein A¹.

In another aspect the invention provides a meat or dairy food product which contains the β-casein A² allele in preference to the β-casein A¹ allele.

In another aspect the invention provides a milk or other dairy product capable of minimising the onset of coronary heart disease characterised in that the milk or other dairy product is substantially free of β-casein A¹, or its proteolytic or heat produced products.

In a further aspect the invention provides a process for producing milk or other dairy products which does not contain β-casein A¹ by testing the individual cows in a herd for the presence of the β-casein A¹ allele, or the presence of β-casein A¹ in their milk, and selectively culling from the herd those cows that test positive for the presence of the β-casein A¹ allele, or β-casein A¹, until the bulk milk produced by the herd is substantially free of β-casein A¹.

In a related aspect the invention provides a process for producing milk or other dairy products which does not contain β-casein A¹ by testing the individual cows in that herd for the presence of the β-casein A¹ allele or β-casein A¹ in their milk and subsequently employing breeding programmes which select against individual cows testing positive for the presence of the β-casein A¹ allele or β-casein A¹ until the bulk milk produced by the herd is substantially free of β-casein A¹.

In a further related aspect the invention provides a process for producing milk or other dairy products which does not contain β-casein A¹ by testing individual cows in a herd for the presence of the β-casein A¹ allele or β-casein A¹ in their milk and utilising genetic engineering procedures to remove the β-casein A¹ allele or inhibit expression of β-casein A¹ therefrom.

In another aspect the invention provides milk and other dairy products which are substantially free of β-casein A¹.

Optionally the dairy product is casein which is substantially free of β-casein A¹. This may be used as a food for animals or humans.

In another aspect the invention provides a process for producing immunoglobulins and other immune response proteins, in cow's milk from animals not possessing the β-casein A¹ allele, or other commercial milk producing animals, to this allele, to counteract the immunosuppressant substances present that are produced from it, in commercial milking cows such as Holsteins, together with its blending with non-treated milk or the recovery of such immunoproteins.

In another aspect the invention provides immunoglobulins and other immunoproteins produced as a result of inoculating commercial milk producing animals with β-casein A¹, its proteolytic hydrolysis products, or fragments thereof produced by other means.

In another aspect the invention provides blended milk obtained from mixing the product of the present invention with milk from animals possessing the β-casein A¹ allele as part of their genetic make-up either at the factory or by running a mixed herd of such animals.

In a related aspect the invention provides an immune milk (as herein described), such immune milk being substantially free of β-casein A¹ and/or produced from cattle or other commercial milking animals lacking the β-casein A¹ allele.

Preferably the immunoglobulins active against β-casein A¹ and its proteolytic products, are recovered by ultrafiltration, ion exchange chromatography or an immunoadsorbent column.

The milk containing immunoglobulins active against β-casein A¹ and its proteolytic products may be in the form of whole milk, whole-milk powder, skim milk, skim milk powder, milk whey, yoghurt, cheese, or any other dairy product, or processed dairy product.

In another aspect the invention provides immunoglobulins produced by treating a homozygous β-casein A², B, or C, or heterozygous mixture of A², B, and C, cow, with any inoculum to produce immunoglobulins in the milk from a cow that does not contain the immunosuppressant β-casein A¹ allele, β-casein A¹, proteolytic fragments thereof or fragments thereof produced by other means.

In a yet further aspect the invention provides a method of reducing the onset of disease in an individual or a population which derives some of its food intake from milk or other dairy products by reducing or substantially eliminating the presence of β-casein A¹ in the diet of that population. This method is applicable to animals or humans.

It is believed that the invention is applicable to reducing the onset of diseases such as:

coronary heart disease, cerebrovascular diseases, duodenal ulcer, peptic ulcer, respiratory diseases, such as bronchitis, lung cancer, asthma's and pneumonia, diabetes, polyarthritis, chronica, Psoriasis, renal disease, systemic lupus erythematosus, chronic disorders of the immune system, and any disease where there is a seasonal variation in incidence or death rate.

Preferably the invention provides a method of reducing the onset of coronary heart disease in a human population which derives some of its food intake from milk or other dairy products by reducing or substantially eliminating the presence of β-casein A¹ in the diet of that population.

In a yet further aspect the invention provides a method of enhancing the immune response or decreasing the immune suppression of an individual or a population who or which derives some of his/her/its food intake from milk or other dairy products by reducing or substantially eliminating the presence of β-casein A¹ in the diet of that individual or that population.

The subject of this invention is the identification of the class of proteins responsible for a number of disorders such as coronary heart disease (and others as described above), their neutralisation in cow's milk and the production of an immunoglobulin capable of partially overcoming some of the deleterious effects they(or it) engender(s) on the human body. This invention is not limited to a specific disease as the molecules concerned appear to act as immunosuppressants to the body's immune system and their removal can only enhance the general well-being of the individual while at the same time providing specific relief to individual's whose genetic make-up is such that contact with these proteins or protein will bring about a specific response such as atherosclerosis or other chronic disorders of the immune system.

The discovery That is the Basis of This Invention

It has been reported that certain groups of peoples are not subject to the diseases described above, notwithstanding the fact that they consume considerable quantities of milk proteins. These people include the Tibetans, rural Gambians, the Masai and Samburu people of Kenya. The latter peoples are also found not to suffer from obesity, even in old age. The only major difference between the milk consumed by the above people is that it is derived from Zebu, Bos Indicus, and Yak, Bos Mutus. Neither milk contains the casein allele described as β-casein A¹. In addition, people such as the Eskimo do not suffer from diseases such as CHD compared with their dairy product consuming Danish countrymen as is illustrated in Table 1:

TABLE 1
Age-adjusted differences in morbidity from chronic diseases between
Greenland Eskimos and Danes

| | Eskimos/Danes |
|---|---|
| Acute myocardial infarction | 1/10 |
| Stroke | 2/1 |
| Psoriasis | 1/20 |
| Diabetes | rare |
| Bronchial asthma | 1/25 |
| Malignant disorders | 1/1 |
| Thyrotoxicosis | rare |
| Multiple sclerosis | 0 |
| Polyarthritis chronica | low |

Acta Med Scand 208: 401-406, (1980)

These and other aspects of this invention, which should be considered in all its novel aspects, will become apparent from the following description, which is given by way of example only with reference to the preferred embodiments, and makes reference also to the following graphs:

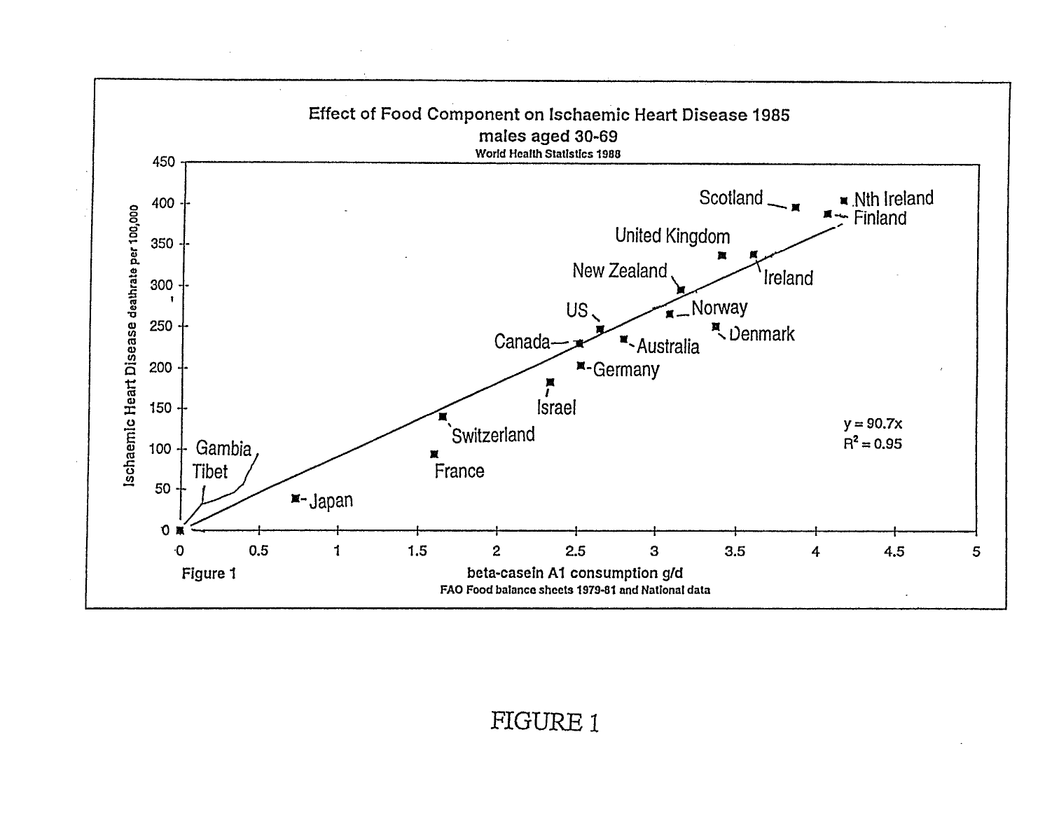

FIG. 1 is a graph entitled “The effect of food component on Ischaemic Heart Disease during 1985 for males aged 30-69”. This shows the death rate of all ages per 100,000 of population, for a range of countries, based on the consumption of β-casein.

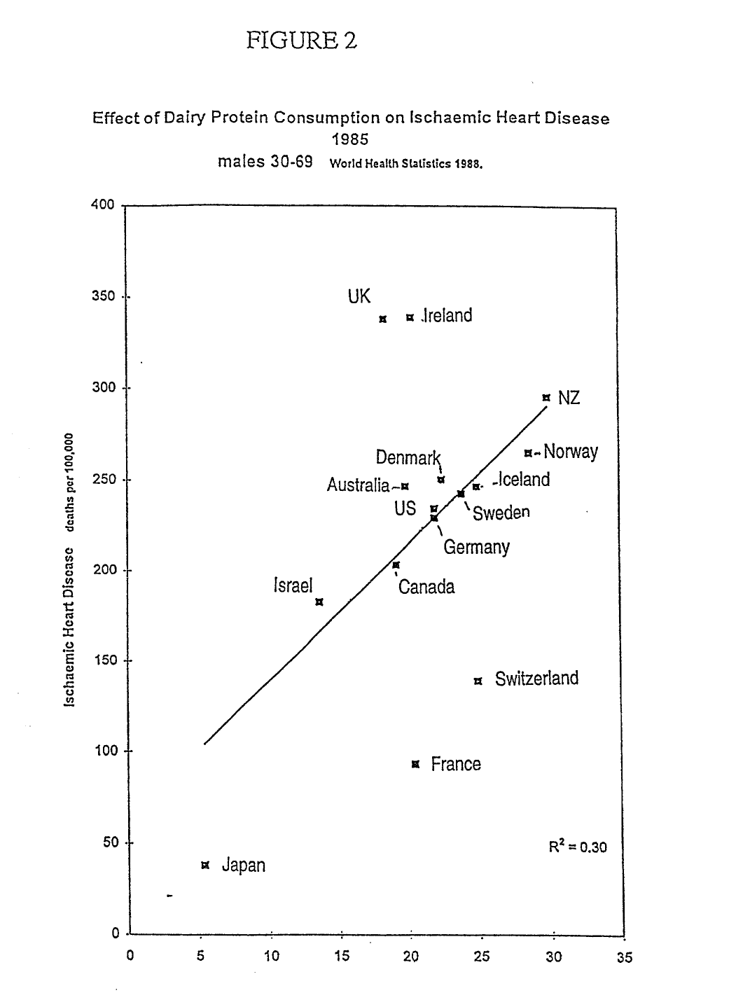

FIG. 2 is a graph showing the effect of dairy protein consumption on Ischaemic Heart Disease for males aged 30-69 for the year 1985.

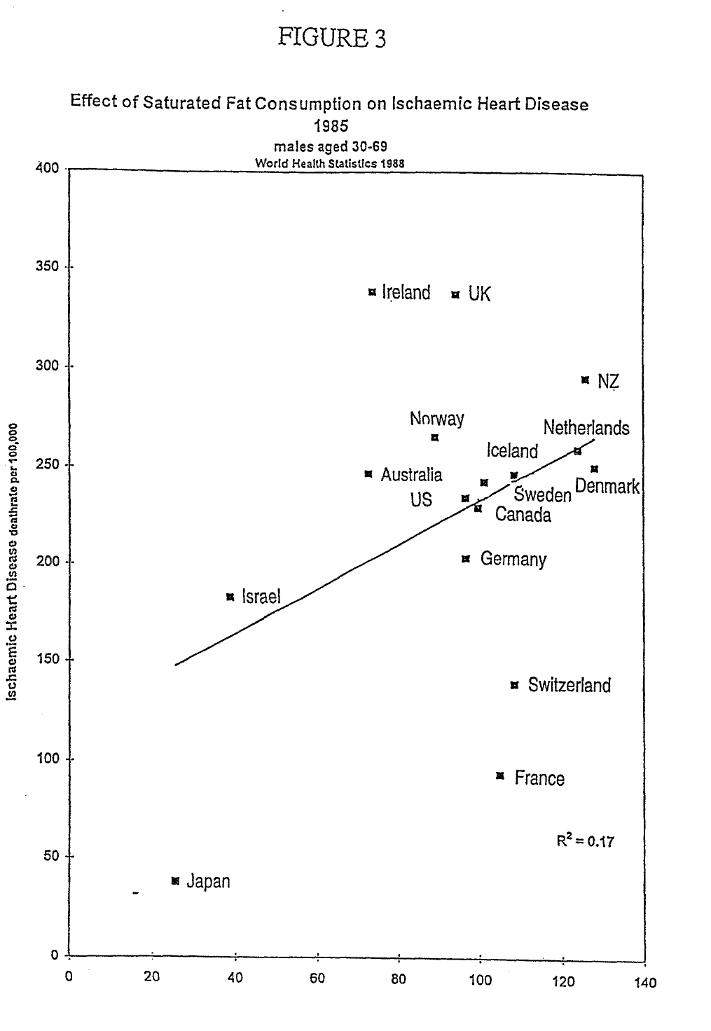

FIG. 3 is a graph showing the effect of saturated fat consumption on Ischaemic Heart Disease for males aged 30-69 for the year 1985.

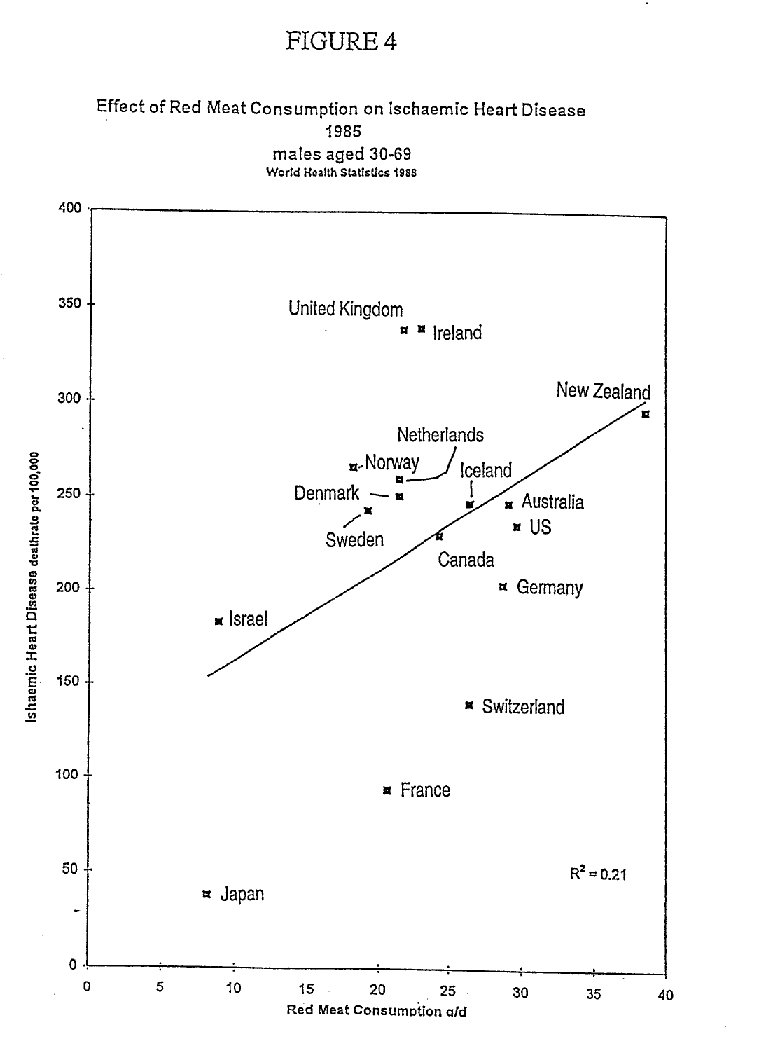

FIG. 4 is a graph showing the effect of red meat consumption on Ischaemic Heart Disease for males aged 30-69 for the year 1985.

FIG. 1 shows a very strong correlation between the consumption of the food component, identified as β-casein A¹ (discussed in more detail below), and the death rate. Whereas the overall dairy protein consumption (FIG. 2) does not provide such a strong correlation nor does the effect of saturated fat consumption (FIG. 3), nor the consumption of red meat (FIG. 4) come anywhere close to the very strong correlation with the inventor has identified in relation to the consumption of β-casein A¹, both between countries and within countries. In the states of the form West Germany Ischaemic Heart Disease death rates are found to correlate directly with the consumption of β-casein A¹ (Table 1A). In this instance the composition of the state dairy herd have remained virtually constant from the 1950's through to the 1980's.

TABLE 1A
CHD nutritional risk factors, Federal Republic of Germany based on
Schleswig-Holstein

| | Satur- | Chole- | Alco- | Carbo- | En- | | Rel |
|---|---|---|---|---|---|---|---|
| | ated Fat | sterol | hol | hydrates | ergy | β-A¹ | IHD est. |
| Schleswig Holstein | 1.00 | 1.00 | 1.00 | 1.00 | 1.00 | 1.0 | 1.0 |
| Niedersachsen | 0.97 | 0.96 | 1.00 | 0.98 | 0.99 | 0.92 | 0.88 |
| Nordrhein Westfalen | 0.99 | 1.02 | 0.99 | 1.00 | 1.02 | 0.97 | 1.00 |
| Hessen | 0.95 | 0.96 | 0.98 | 0.98 | 0.98 | 0.75 | 0.74 |
| Rheinland-Pfalz | 0.95 | 0.99 | 1.00 | 1.02 | 1.0 | 0.87 | 0.78 |
| Saarland | 0.94 | 0.93 | 0.98 | 1.01 | 0.98 | 0.90 | 0.88 |
| Baden Wurttenburg | 0.93 | 1.02 | 1.02 | 1.05 | 1.03 | 0.50 | 0.72 |
| Bayern | 0.96 | 0.99 | 1.22 | 1.06 | 1.02 | 0.50 | 0.74 |

### DETAILED DESCRIPTION

Caseins constitute the majority of the milk proteins. Dairy cattle exhibit genetic polymorphism in their proteins. The heterogeneity of the caseins is further complicated by the fact that they are the products of co-dominant allele autosomal genes. Some indication of their number, and the major product fragments into which they are split by proteolytic action of a variety of enzymes, is illustrated by the β-caseins in Table 2.

TABLE 2
The β-casein family of proteins

| Former nomen. | recommended nomen. | source of fragment |
|---|---|---|
| β-casein A¹ | β-CN A¹-5P | — |
| β-casein A² | β-CN A²-5P | — |
| β-casein A³ | β-CN A³-5P | — |
| β-casein B | β-CN B-5P | — |
| β-casein C | β-CN C-4P | — |
| β-casein D | β-CN D-4P | — |
| β-casein E | β-CN E-5P | — |
| γ1-casein A¹ | β-CN A¹-1P(f29-209) | β-CN A¹-5P |
| γ1-casein A² | β-CN A²-1P(f29-209) | β-CN A²-5P |
| γ1-casein A³ | β-CN A³-1P(f29-209) | β-CN A³-5P |
| γ1-casein B | β-CN B-1P(f29-209) | β-CN B-5P |
| γ2-casein A² | β-CN A² (fl06-209) | β-CN A¹-5P or β-CN A²-5P |
| γ2-casein A³ | β-CN A³ (fl06-209) | β-CN A³-5P |
| γ3-casein B | β-CN B (fl06-209) | β-CN B-5P |
| γ3-casein A | β-CN A (fl08-209) | β-CN A¹-5P, β-CN A²-5P or |
| | | β-CN A³-5P |
| γ3-casein B | β-CN B (fl08-209) | β-CN B |

In addition there are a number of proteose peptone components.
W. N. Eigel Nomenclature of Proteins of Cow's Milk: Fifth Revision
Dairy Science 67:1599-1631, (1984)

Most animals are heterozygous, That is their protein composition contains a mixture of the various alleles inherited from the genes of their sire and dan. It appears that the original cow from which the current domesticated species developed contained only the β-casein A² allele. β-casein A¹ differs from A² in containing the following amino acid substitution, proline at position 67 is replaced with histidine. The corresponding A¹ allele is a relatively recent modification. However some animals are homozygous, that is their proteins are of one type only; in the case of β-caseins either A¹, A², A³, or B, C, D or E.

Bovine milk is an important source of proteins and other nutrients required by humans &id the common domestic cattle species such as the Holstein have greater quantities of the A¹ allele than any other β-casein allele. Approximately 84 percent of the present American dairy herd is estimated to carry this allele.

In the graph shown in FIG. 1 the consumption of β-casein A¹ (and its derived proteolysis products) are plotted against the incidence of ischaemic heart disease based on FAO Food Balance Sheets 1979-81 and WHO Trends in Mortality for Selected Causes of Death 1985-1989 and other reported CHD data.

In Table 3 the effect of heating milk to 63° C. for 20-30 minutes, known as Holder Pasteurisation, is set out together with the corresponding rate of CHD.

TABLE 3
CHD rates following the Introduction of Holder Pasteurisation

| | | Angina pectoris (AP1) | | | | Cerebral embolism | | | |
|---|---|---|---|---|---|---|---|---|---|
| Population | Holder intro. | mort. p mill. | | | | and thrombosis (CET) | | | |
| group | year | AP1 | AP2 | AP3 | Δ% | CET 1 | CET 2 | CET 3 | Δ% |
| U.K | | | | | | | | | |
| Edinburgh | 1923 | 1925 | 67 | 92 | 37.3^{a} | 1924 | 174 | 236 | 35.6 |
| Glasgow | 1924 | 1924 | 56 | 91 | 62.5^{a} | 1924 | 77 | 101 | 31.2 |
| Dundee | 1924 | 1925 | 42 | 64 | 52.4^{a} | 1925 | 162 | 188 | 16.0 |
| Aberdeen | 1926 | 1926 | 91 | 135 | 48.4^{a} | 1927 | 121 | 227 | 87.6 |
| Lanarkshire | 1935 | 1937 | 188 | 375 | 99.5^{b} | 1938 | 153 | 193 | 26.1 |
| (excluding | 1947 | 1948 | 685 | 1185 | 73.0 | 1948 | 298 | 518 | 73.8 |
| Glasgow) | 1952 | 1954 | 1185 | 1523 | 28.5 | 1954 | 518 | 680 | 31.3 |
| County of | 1954 | 1954 | 963 | 1710 | 77.9 | 1954 | 610 | 823 | 34.9 |
| Sutherland | | | | | | | | | |
| County of | 1956 | 1956 | 1610 | 2848 | 76.9 | 1956 | 955 | 1398 | 46.4 |
| Bute | | | | | | | | | |
| London Admin. | 1925 | 1925 | 31 | 112 | 261.3^{c} | 1926 | 90 | 120 | 33.3 |
| County | | | | | | | | | |

| | | | |
|---|---|---|---|
| | Average increase | 81.8 | 41.6 |
| Norway | | | |

| | | | | | | |
|---|---|---|---|---|---|---|
| Oslo | 1922 | 1922 | 3 | 43 | 1333.3^{d} | not available |

Columns AP1 and CET1 denote the year of commencement of the sudden rise in the appropriate mortality.
Columns AP2 and CET2 denote the appropriate average mortality for the 4 years immediately preceding the year of introduction of pasteurisation.
Columns AP3 and CET3 denote the appropriate average mortality for the 4 years immediately succeeding the introduction of pasteurisation.
Δ% represents average increase.
^{a}Possibly low because deaths ascribed to “coronary thrombosis” were not included in International List No. 89 in Scotland until 1931.
^{b}Possibly enhanced as deaths ascribed to “coronary thrombosis” were now included in International List No. 94.
^{c}Possibly enhanced because (I) after 1927 all deaths ascribed to “coronary thrombosis” were included-unlike those in Scotland-in International list No. 89 and (ii) the large London creameries introduced Holder pasteurisation during this period.
^{d}Mortality ascribed to the following group of classifications: angina pectoris, infarctus cordis, sclerosis art. coron. cordis.

A proteolytic enzyme plasmin, which is naturally present in milk, and which is largely associated with the casein, is both increasingly active at higher temperatures and is quite heat stable. At 60° C. it has been demonstrated to have a relatively high rate of conversion of caseins, preferentially β-caseins to a range of proteolysis products. The increased mortality rate, demonstrated in Table 3, as a result of heating of the milk is presumed to be due to the formation of further proteolysis products, in addition to those naturally present, during the heating phase.

It is possible however that the specific fragment of β-casein A¹ that is entering or effecting the body's immune system which result from an enzyme contained within a psychotropic bacterium, or spore forming bacterium, present in the milk. Both the ratio of β-casein A¹/β-casein A² and the concentration of psychotropic bacteria vary seasonally in milk. This seasonal fluctuation is thought responsible for part of the seasonal fluctuation in the illnesses that we have noted above.

This work is further supported by the results of Bell Rc, Golemboski K A, Dietert R R, and Campbell T C, Nutrition and Cancer 22;(2),151-162,(1994) who found that when Fischer 344 rats were fed diets containing 6 percent and 22 percent casein after being injected with a liver cancer causing substance, aflatoxin, the percentage of animals developing liver cancer increased directly proportional to the increase in casein in the diet. They interpreted the results to suggest that a low protein diet might result in lower suppression of the natural killer cell cytotoxicity activity. With our knowledge we can re-interpret their data to suggest that based on our own observations on the effect of β-casein A¹ on immunosuppression in humans, its reduction in the rat's diet reduced cancer formation by a factor of four due to a dose specific effect on the rat's immune system.

The preferred forms of this invention comprises the elimination from milk of β-casein A¹ or its proteolysis products, or protein fragments formed in any other way, either ‘in vitro’ or ‘in vivo’ and which have immunosuppressant properties, by the use of immunoglobulins raised against β-casein A¹, the removal of β-casein A¹ and the inactivation of plasmin and other proteolytic enzymes. The preferred forms of the invention represents a significant advance over existing treatments for atherosclerosis, and other generally chronic immunosuppressant diseases in that it will prevent their occurrence in the new-born who, when they are genetically susceptible, will in other circumstances develop the diseases as they age. In addition it is believe it will assist in the restoration of organs and cells in those people where the damage to the bodies' organs is not permanent, by removing the source of chronic immune suppression.

By the term treatment, for the purpose of this invention it is intended that the symptoms of the disorder be ameliorated or completely eliminated or, where genetic typing indicates that an individual is of high risk of developing a disorder, of ensuring that it does not develop.

### EXAMPLE 1

In its preferred form the treatment consists of inoculating a milk producing animal, that does not possess the β-casein A¹ allele, preferably one that is homozygous for the β-casein A² allele, preferably one that produces commercially feasible quantities of milk, such as a cow, sheep, goat, or zebu with β-casein A¹, or its proteolysis products, or fragments thereof, produced in any other manner, so that antigens to the foreign β-casein A¹ protein are produced. These antigens may be produced either alone, or as part of a wider inoculation programme, to produce a milk with an enhanced antigen concentration as has been described in the Art. This antigen enhanced milk is then added to ‘normal’ milk, or milk, or milk products, whose β-casein A¹ content has been reduced, using techniques known to those skilled in the Art, to counteract the presence of the immune suppressing β-casein A¹ derived material. Alternatively the above antigen(s) may be recovered by one of the processes known to the Art and used as a food supplement in its own right either alone or as a food additive.

Because the immunoglobulins formed as a result of the inoculation programme are somewhat heat sensitive then care has to exercised with the pasteurisation and handling of the final product if a powder is required as is described in the existing Art.

Alternatively, immunoglobulins and other antigens, are recovered from non β-casein A¹ containing milk by ultrafiltration, ion exchange chromatography either singly, or in combination, or by use of a suitable immunoadsorbent column, comprising an insoluble carrier material to which is bound a low-affinity monoclonal antibody specific to one or more milk immunoglobulins but not specific to any other common constituent of milk. Such milk may having been derived from an animal that has been inoculated with a vaccine derived from a bacteria such as E. coli, for example, or which has been inoculated with ‘bacterial antigens’, as described in the Art. Alternatively, enhanced quantities of antigens are produced as a result of the inoculation, or inoculation programme of β-casein A¹-free animals, to provide a milk product with all the claims as described in the prior Art. This invention has the advantage over the existing Art that immunosuppressant proteins resulting from the presence of, or, derived from the β-casein A¹ allele are eliminated from the final milk, or milk-derived products.

Another alternative includes the use of a plasmin inhibitor, such as a protein like aprotonin, or other such inhibitors, known to the Art, which are added, either singly or in mixtures, to the milk, as part of the above invention, to suppress the formation of additional β-casein A¹ proteolysis products that would otherwise be formed during processing, and storage, prior to sale.

### EXAMPLE 2

A milk or other dairy product according to the invention can be produced by testing individual cows in a dairy herd for the presence of the β-casein A¹ allele, or for the presence of β-casein A¹ in milk, and then selectively culling those cows returning a positive result, until the bulk milk produced by the herd is substantially free of β-casein A¹. Alternatively, homozygous cattle containing the β-casein A² allele can be selectively bred so that the β-casein A¹ allele is eliminated from the herd.

An alternative approach to remove β-casein A¹ from bulk milk would involve separating cattle from existing herds which contain the β-casein A¹ allele, allowing the remainder of the herd (which are free of the β-casein A¹ allele) to be used for the production of bulk milk or other dairy products, and those cattle containing the A¹ allele to be used for the production of products for purposes other than human consumption. Such a segregation process within a herd may be facilitated by the use of ear tags or the like to mark individual animals.

### Industrial Application

The invention provides a useful food product capable of increasing the health of an individual, or the health of a population. In one aspect the invention provides a method of enhancing the immune response of an individual or a population who or which derives some of his/her/its food intake from milk or other diary products, by reducing or substantially eliminating the presence of β-casein A¹ in the diet of that individual or that population.

In a particularly preferred form, the invention applies a method of reducing the onset of coronary heart disease in a human population which derives some of its food intake from milk or other diary products by reducing or substantially eliminating the presence of β-casein A¹ within the diet of that population.

### Advantages

By reducing or substantially eliminating the presence of β-casein A¹ in the diet of humans, it is believed that the immune response of an individual or a population may be enhanced, or immunosuppression reduced, increasing the general well-being of the individual or the population. It is believed that some individuals may be particularly susceptible to the presence of β-casein A¹, and it may be possible to develop a test for such susceptible individuals, and to recommend that they reduce or eliminate their consumption of milk or other diary products containing β-casein A¹.

### Variations

Recognising that dairy products free of β-casein A¹ are desirable it is preferable to ensure that the animal from which the product is derived has been tested for the presence of the β-casein A¹ allele or β casein A¹ expressed therefrom and subsequent selective breeding programmes (selecting for β-casein A¹ negative animals) carried out to eliminate the presence of the β-casein A¹ from the herd. It will be recognised that such testing may be carried out in a number of ways without departing from the scope of the present invention.

Without departing from the scope of this invention an alternative approach to remove the β-casein A¹ allele from a herd may be carried out. Such an approach may include the screening of sperm to be used for the purpose of artificial insemination for the presence or absence of the β-casein A¹ allele and selecting against those sperm which contain this allele.

In addition to the methods of removing β-casein A¹ (or the β-casein A¹ allele), from meat products, milk and “processed dairy products” that have been disclosed herein it would be within the scope of this invention to use a number of alternative methods. Such alternative methods may involve the removal of β-casein A¹ from milk products via ultrafiltration techniques or by utilising a non-toxic chemical or enzymatic process to remove or inactivate β-casein A¹.

Finally, it will be appreciated that various other alterations and modifications may be made to the foregoing without departing from the spirit or scope of this invention.

What is claimed is:

1. A method of preparing a product suitable for human consumption that is free of beta-casein A¹ and derived from a herd of cattle, the method comprising the steps of:
testing individuals from the herd for the presence or absence of the beta-casein A¹ allele or beta-casein A¹ or proteolytic fragments thereof;
selecting those individuals from the herd that are free of the beta-casein A¹ allele or free of beta-casein A¹;
obtaining raw products from the selected individuals;
combining the raw products of at least two of the selected individuals; and
using the combined raw products to prepare a product suitable for human consumption.

2. The method according to claim 1, further comprising the step of testing the combined raw materials for the presence or absence of beta-casein A¹.

3. A product suitable for human consumption that is free of beta-casein A¹ and prepared from a herd of cattle consisting of individuals which have been tested for the presence of beta-casein A¹ allele, beta casein A¹, or proteolytic fragments thereof and those individuals testing positive having been culled from the herd, or individuals derived from at least one breeding pair of cattle which have been tested for the presence of the beta-casein A¹ allele, beta-casein A¹, or proteolytic fragments thereof and are beta-casein A¹ free.

4. The product as claimed in claim 3, wherein said product is bulk milk.

5. The product is claimed in claim 4, wherein the bulk milk is pasteurized.

6. The product as claimed in claim 3, wherein said product is a processed dairy product obtained from bulk milk.

7. The product as claimed in claim 3, wherein said product is casein derived from bulk milk.

8. The product as claimed in claim 3, wherein said product is an immune milk derived from bulk milk.

9. The product as claimed in claim 3, wherein said product is a blood or meat product.

10. A herd of cattle consisting of individuals which have been tested for the presence of the beta-casein A¹ allele, beta-casein A¹, or proteolytic fragments thereof and those individuals testing positive having been culled from the herd, or individuals derived from at least one breeding pair of cattle which have been tested for the presence of the beta-casein A¹ allele, beta-casein A¹, or proteolytic fragments thereof and are beta-casein A¹ free.
